(19) United States
(12) Patent Application Publication
(10) Pub. No.: US 2024/0095455 A1
(43) Pub. Date: Mar. 21, 2024

(54) SYSTEMS AND METHODS FOR QUESTION-ANSWERING USING A MULTI-MODAL END TO END LEARNING SYSTEM

(71) Applicant: CADENCE SOLUTIONS, INC., New York, NY (US)

(72) Inventors: Ashwyn SHARMA, Seattle, WA (US); David I. Feldman, Miami, FL (US)

(73) Assignee: CADENCE SOLUTIONS, INC., New York, NY (US)

(21) Appl. No.: 17/817,574

(22) Filed: Aug. 4, 2022

Related U.S. Application Data
(63) Continuation of application No. 17/812,524, filed on Jul. 14, 2022, now abandoned.

(51) Int. Cl. G06F 40/30; G06F 40/103; G06F 40/166; G06F 40/279; G06V 30/19; G16H 10/60

(52) U.S. Cl. CPC G06F 40/30; G06F 40/103; G06F 40/166; G06F 40/279; G06V 30/19; G16H 10/60

(57) ABSTRACT

A multi-modal end to end learning system configured to answer questions about clinical documents like patient notes, medical reports, and lab results. Documents are polled from an electronic medical record system, converted to text, and scrubbed for protected health information before processing. Sanitized text data is then fed as context to a language model that has been fine-tuned for question-answering (QA). The other input to the model is a prompt or a question that is either provided on-the-fly by a clinician as part of a search or pre-determined for specific needs. In return, the model outputs an answer highlighting part of the text/image where it found the answer and a confidence score quantifying the likelihood of the answer being correct. A clinician can optionally correct the answer if needed. This feedback by the clinician is fed back to a fine-tuner module and used to improve the model over time.

### CROSS-REFERENCE TO RELATED APPLICATIONS

This application is a continuation of U.S. application Ser. No. 17/812,524 filed Jul. 14, 2022, which is incorporated by reference in its entirety.

### BACKGROUND

Search algorithms have evolved considerably over time. Early search algorithms relied on methods that matched a query's keywords with documents that contain the most mentions of the keywords. These algorithms would then return a list of results to a user in the form of search results. Search algorithms have continued to evolve, and the advent of semantic search has led to advancements in the ways in which computers understand and provide results for human generated queries.

However, some industries, for example the medical field, have not seen the same marked improvement in searching techniques due to the confidential nature of health records and the esoteric industry-specific terms used by medical professionals. For example, while clinical documents like patient notes, medical reports, and lab results constitute high-quality, high-dimensional data sources, clinical documents are heavily under-utilized by clinical data analyses, because it is challenging to make sense of natural language text and images in a field that heavily relies on a very domain-specific lexicon.

Accordingly, there is a need for systems and methods that overcome the deficiencies of prior art systems by employing advanced algorithms that can understand the semantic structure of user queries and can further provide precise results to said queries.

### SUMMARY

The instant system and methods provide a multi-modal end to end learning system that intelligently answers questions about clinical documents like patient notes, medical reports, and lab results. The system may leverage an extractive question answering (QA) natural language processing model to provide both a specific answer and a relevant portion of a section of a document in response to a user query.

The multi-modal end to end learning system may poll documents stored in a secure cloud-based electronic medical record system via a task scheduler on a periodic basis. The polled documents may be converted to text and scrubbed (i.e., cleaned and sanitized) for protected health information before being processed. Documents that are in image format are converted to text using an optical character recognition model and both the image and the text within the image are separately stored. Documents that are text-based have the text extracted and stored. In both instances, the text gleaned from the document is cleaned and sanitized, and then fed as context to a language model that has been fine-tuned for extractive question-answering. In addition to the cleaned and sanitized text data, a prompt or question—that is either provided on-the-fly (i.e., in real-time) by a clinician as part of a search or is pre-determined for specific needs—is also fed as input to the extractive QA language model. In return, the extractive QA language model outputs an answer to a user device highlighting part of the document/image wherein the answer was found and a confidence score quantifying the likelihood of the answer being correct. Subsequently, a user (e.g., a clinician) operating the user device may provide feedback regarding the answer that was provided and said feedback may be used to fine-tune the extractive QA language model.

In one embodiment the multi-modal end to end learning system may include a server comprising one or more processors and a non-transitory memory, in communication with the server, storing instructions that when executed by the one or more processors, cause the one or more processors to implement a method comprising: receiving one or more documents, converting the one or more documents to a second format via one or more computer vision techniques, wherein text from the document is stored in a corpus of text, receiving a prompt from a user device, feeding the corpus of text and the prompt as input into a natural language processing model, wherein the corpus of text serves as context for the natural language processing model, determining an answer to the prompt via the natural language processing model, transmitting the answer to the user device, receiving feedback associated with the answer from the user device, and fine-tuning the natural language processing model using that feedback.

In another embodiment the multi-modal end to end learning system may include a computer-implemented method comprising: receiving one or more documents, converting the one or more documents to a second format via one or more computer vision techniques, wherein text from the document is stored in a corpus of text, receiving a prompt from a user device, feeding the corpus of text and the prompt as input into a natural language processing model, wherein the corpus of text serves as context for the natural language processing model, determining an answer to the prompt via the natural language processing model, transmitting the answer to the user device, receiving feedback associated with the answer from the user device, and fine-tuning the natural language processing model using that feedback.

In another embodiment the multi-modal end to end learning system may include non-transitory computer-readable medium storing instructions, that when executed by one or more processors, cause the one or more processors to implement the instructions for: receiving one or more documents, converting the one or more documents to a second format via one or more computer vision techniques, wherein text from the document is stored in a corpus of text, receiving a prompt from a user device, feeding the corpus of text and the prompt as input into a natural language processing model, wherein the corpus of text serves as context for the natural language processing model, determining an answer to the prompt via the natural language processing model, transmitting the answer to the user device, receiving feedback associated with the answer from the user device, and fine-tuning the natural language processing model using that feedback.

Notably in some of the previously discussed embodiments, converting the document to a second format further comprises redacting protected health information from the document.

In some of the previously discussed embodiments, the natural language processing model is an extractive QA model, and the natural language processing model is pre-trained on electronic medical records. In addition, the natural language processing model is fine-tuned using feedback from the user device.

In some of the previously discussed embodiments, determining an answer further comprises evaluating multiple possible answers included in the corpus of text via one or more metrics that: compares the text in the prompt to the multiple possible answers to identify an exact match, or applies a weighted average of precision and recall of each of the multiple possible answers.

In some of the previously discussed embodiments, transmitting the answer to the user device further comprises generating instructions to visibly highlighting and providing the passage of the document where the answer is located.

### BRIEF DESCRIPTION OF THE FIGURES

FIG. 1 illustrates a computing environment, according to various embodiments of the present disclosure.

FIG. 2 illustrates a multi-modal end to end learning system framework, according to various embodiments of the present disclosure.

FIG. 3 illustrates a fine-tuner workflow diagram, according to various embodiments of the present disclosure.

FIG. 4 illustrates a method for extractive question answering, according to various embodiments of the present disclosure.

FIG. 5 illustrates a block diagram for a computing device, according to various embodiments of the present disclosure.

### DETAILED DESCRIPTION

Embodiments of the present disclosure relate to systems and methods for extractive QA with clinical data using a multi-modal end to end learning system comprised of artificial intelligence/natural language processing models. The implementation of these novel concepts may include, in one respect, fine-tuning a language model on extractive QA techniques. In another respect, these novel concepts may include using a prompt (e.g., a natural language question and/or a user query) and sanitized clinical text and documents as inputs for the language model. In another respect, these novel concepts may include the language model generating output including an answer to the prompt and highlighting the portion of the text within a document where the answer was found.

The disclosed principles are described with reference to clinical documents and processing by an electronic medical record system, but it should be understood that these principles may apply to any type of document requiring processing and/or a response by a recipient of the document and any electronic service or system that processes or uses said documents. Accordingly, the disclosed principles are not limited to use with clinical documents.

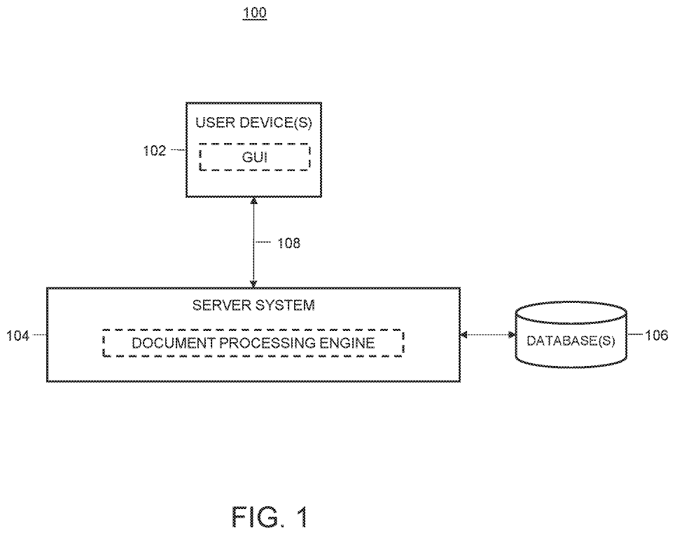

Referring to FIG. 1, computing environment 100 may be configured to automatically and intelligently process documents such as clinical documents, according to embodiments of the present disclosure. Computing environment 100 may include one or more user device(s) 102, a server system 104, and one or more databases 106 communicatively coupled to the server system 104. The user device(s) 102, server system 104, and database(s) 106 may be configured to communicate through network 108.

In one or more embodiments, user device(s) 102 is operated by a user (e.g., a clinician) and may be a device configured to communicate with an electronic medical records system. User device(s) 102 may be representative of a mobile device, a tablet, a desktop computer, or any computing system having the capabilities described herein. Users may include, but are not limited to, individuals such as, for example, clinicians, companies, prospective clients, patients, and/or customers of an entity associated with server system 104, such as individuals who have received and or produce clinical documents and are utilizing the services of, or consultation from, an entity associated with that document and server system 104.

User device(s) 102 according to the present disclosure may include, without limit, any combination of mobile phones, smart phones, tablet computers, laptop computers, desktop computers, server computers or any other computing device configured to capture, receive, store and/or disseminate any suitable data. In one embodiment, a user device(s) 102 includes a non-transitory memory, one or more processors including machine readable instructions, a communications interface which may be used to communicate with the server system (and, in some examples, with the database(s) 106), a user input interface for inputting data and/or information to the user device and/or a user display interface for presenting data and/or information on the user device. In some embodiments, the user input interface and the user display interface are configured as an interactive graphical user interface (GUI). The user device(s) 102 are also configured to provide the server system 104, via the interactive GUI, input information (e.g., documents such as e.g., questions, prompts, queries, patient records, patient charts, lab results, clinician notes, and diagnoses) for further processing. In some embodiments, the interactive GUI is hosted by the server system 104 or provided via a client application operating on the user device. In addition, the interactive GUI may include multiple distinct regions where results can be provided, feedback can be inputted, and clinical documents can be created, updated, and revised. In some embodiments, a user operating the user device(s) 102 may query server system 104 for information related to a received document (e.g., a clinical document).

Server system 104 hosts, stores, and operates a document processing engine, or the like, for automatically and intelligently processing documents for, and training of, a multi-modal end to end learning system. For example, if the server system 104 supports or provides an electronic medical records (EMR) system, it will include the capability to process documents such as clinical documents.

The document processing engine may asynchronously monitor, retrieve according to a schedule, and enable the submission of documents (e.g., clinical documents) received by the user device(s) 102. The server system 104, in response to receiving the one or more documents, may store images and text included in the one or more documents, in one or more storage devices. Server system 104 may additionally convert the one or more documents to a computer interpretable format via one or more computer vision techniques and extract text from the document. Server system 104 may additionally redact protected health information from the one or more documents. In one or more embodiments, during the redaction process, server system 104 removes predetermined objects such as patient: name, address, social security number, images, unique identifying characteristics, and the like. Server system 104 may then feed the text data gleaned from the one or more documents as context for a language model. The server system 104 may receive input from a user, for example, a question, query, and/or prompt, which the server system 104 additionally feeds to the language model as input.

The language model leveraged by server system 104 may be fine-tuned for QA. The language model may be configured to analyze the received context and prompt; and intelligently identify an answer to the prompt, the location of the answer within one or more documents, and generate a confidence score indicating the probability of the answer being accurate.

Server system 104 may transmit the answer to the prompt along with the location of the answer highlighted in the one or more documents, and the confidence score, to a user device. The server system 104 may receive feedback indicative of an acknowledgement as to whether the provided answer was accurate from the user device. Server system 104 may leverage the feedback as input for a fine-tuner that improves the language model over time.

The server system 104 may further generate instructions for displaying documents (or portions of documents) stored in the training dataset and actions that can be taken with said documents, via a GUI that operates on the user device(s) 102. The aforementioned techniques provide accurate and automated solutions that improve upon prior methods for analyzing clinical documents.

The server system 104 may be further configured to implement two-factor authentication, Secure Sockets Layer (SSL) protocols for encrypted communication sessions, biometric authentication, and token-based authentication. The server system 104 may include one or more processors, servers, databases, communication/traffic routers, non-transitory memory, modules, and interface components.

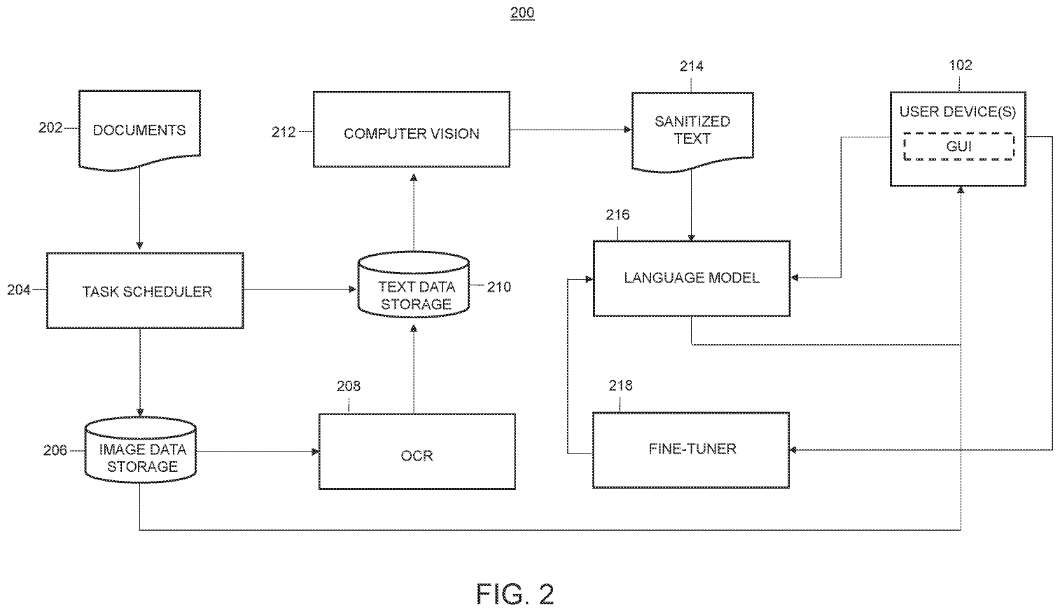

Database(s) 106 may be locally managed and/or a cloud-based collection of organized data stored across one or more storage devices and may be complex and developed using one or more design schema and modeling techniques. In one or more embodiments, the database system may be hosted at one or more data centers operated by a cloud computing service provider. The database(s) 106 may be geographically proximal to or remote from the server system 104 configured for data dictionary management, data storage management, multi-user access control, data integrity, backup and recovery management, database access language application programming interface (API) management, and the like. The database(s) 106 are in communication with the server system 104 and the user device(s) 102 via network 108. The database(s) 106 store various data, including one or more tables, that can be modified via queries initiated by users operating user device(s) 102. In one or more embodiments, various data in the database(s) 106 will be refined over time using a natural language processing model, for example the language model discussed below with respect to FIGS. 2-4. In one or more embodiments, database(s) 106 additionally store training data and historical training data used to refine the language model. Additionally, the database system may be deployed and maintained automatically by one or more components shown in FIG. 1.

Network 108 is any suitable network, including individual connections via the Internet, such as cellular or Wi-Fi networks. In some embodiments, network 108 connects terminals, services, and mobile devices using direct connections, such as radio frequency identification (RFID), near-field communication (NFC), Bluetooth™, low-energy Bluetooth™ (BLE), Wi-Fi™, ambient backscatter communication (ABC) protocols, universal serial bus (USB), wide area network (WAN), local area network (LAN), or the Internet. Because the information transmitted may be personal or confidential, security concerns may dictate one or more of these types of connection be encrypted or otherwise secured. In some embodiments, however, the information being transmitted may be less personal, and therefore, the network connections may be selected for convenience over security.

For example, network 108 may be the Internet, a private data network, virtual private network using a public network and/or other suitable connection(s) that enables components in computing environment 100 to send and receive information between the components of computing environment 100.

Referring to FIG. 2, a multi-modal end to end learning system framework 200 is depicted, according to various embodiments of the present disclosure. Framework 200 provides components and processes for evaluating a document (e.g., a clinical document) and providing QA using one or more natural language processing models. These features provide an improvement over the prior art which merely typically provide keyword-based search results in response to prompts. As shown, framework 200 includes a task scheduler component 204 (e.g., cronjob), which implements a task scheduler configured to schedule tasks to run periodically, at preset times, dates, and/or intervals. Framework 200 additionally includes a computer vision component 212 configured and capable of receiving documents 202 (e.g., a clinical document that may either be an image or document with text) that may include protected health information (PHI), and redacting, removing, and/or obfuscating the PHI such that the remaining text is a different version than what was originally received. This results in a clean and sanitized version of the originally received text. Notably, the data and text in documents 202 may be unstructured, and therefore may not be arranged according to a pre-set model such that the data and text can be processed and analyzed via conventional data tools and methods.

In one embodiment the document may be an image and server system 104 may store the image data in image data storage 206. Server system 104 may then perform optical character recognition (OCR) via the OCR component 208 to convert the document from a first format (e.g., an image) to a second format readable by a computer. Server system 104 may then extract the text recognized from the OCR component 208 and store it in text data storage 210. Alternatively, in another embodiment the document may include text and server system 104 extracts the text and stores it in text data storage 210. In both instances, the text stored in text data storage 210 may be analyzed via a computer vision component 212 that is configured to redact any protected health information recited in the document thereby creating sanitized text 214.

Language model component 216 may implement a language model configured for interpreting a text within a document (e.g., a clinical document) and producing word embeddings for one or more tasks including, but not limited to QA. Language model component 216 may be particularly configured for implementing a language model that can interpret medical terminology, medical codes (e.g., International Classification of Diseases (ICD) codes), medical diagnoses, handwritten medical notes, prescription data, data on charts, images, x-rays, and the like. Language model component 216 may additionally be configured to make associations between text within a document and known acronyms.

Server system 104 may then feed the sanitized text 214 (as context) to language model component 216 as input. Language model component may implement an extractive QA language model. The extractive QA language model may be trained using framework 200, which includes a training dataset (e.g., training dataset 302). Training dataset 302 is a corpus comprising numerous documents (e.g., clinical documents including patient records like charts, diagnoses, medical test results, prescription information, clinician notes, and the like) that may or may not have been previously run through the extractive QA language model component 216.

Framework 200 may additionally include a pre-trained language model component (not shown). A pre-trained language model component may be a deep learning model that is trained on the training dataset 302 and/or one or more other dataset (e.g., a reading comprehension question and answer dataset like SQuAD). The training dataset may be indexed and used by the language model component 216 as context. The training dataset 302 may be comprised of vectors (i.e., context vectors) that can be analyzed and used by one or more downstream processes, for example (as sanitized text 214, which is also referred to as context) input that is referenced when a user's question (e.g., query) is submitted to server system 104. In addition, or alternatively, server system 104 may generate its own synthetic training data via one or more supervised, self-supervised, or unsupervised models.

By incorporating the pre-trained language model component, framework 200 may improve its accuracy and reduce the amount of training time required to perform QA tasks. The pre-trained model language component may include NLP models including but not limited to: Named Entity Recognition (NER), which is a NLP task where the model tries to identify the type of every word/phrase which appears in the input text; sentiment analysis, which is a NLP task where a model tries to identify if the given text has positive, negative, or neutral sentiment; machine translation, which is a NLP task where a model tries to translate sentences from one language into another; text summarization, which is a NLP task where a model tries to summarize the input text into a shorter version in an efficient way that preserves all important information from the input text; natural language generation, which is a NLP task where the model tries to generate natural language sentences from input data or information given by NLP developers; speech recognition, which is a NLP task where a model tries to identify what the user is saying; content moderation, which is a NLP task where a model tries to identify the content that might be inappropriate (offensive/explicit), or should not be shown on public channels like social media posts and/or comments; and automated QA systems: Automated QA systems that try to answer user-defined questions automatically by looking at the input text.

A pre-trained language model component may be trained to understand the grammatical and semantic structure of the corpus composed of clinical documents and medical lexicon. The pre-trained language model component may be trained for days, weeks, or months to accurately understand the medical domain specific language.

The trainer component (not shown) may be a training engine configured to loop over the training dataset and update model parameters. The trainer component may receive the training dataset 302 and the pre-trained language model component as input for one or more training models such as Bidirectional Encoder Representations from Transformers (BERT), Generative Pre-trained Transformer 2 (GPT2), and/or Robustly Optimized BERT Pre-training Approach (RoBERTA). The trainer component may train and modify a language model implemented by the language model component 216 based on the aforementioned input, models and parameters. Notably these models can be used for information retrieval and reading comprehension modules.

The language model component 216 may leverage both the context from the sanitized text 214 and a user's question as input. In turn, language model component 216 may refer back to the context and make predictions about where the answer is inside the one or more documents. In furtherance of identifying a span (i.e., a section and/or passage, which may be visibly highlighted when presented to a user) of a document where the answer is located, the language model component 216 may generate a confidence score associated with the prediction that the provided answer and identified span of text is accurate. Notably, the extractive QA language model implemented by language model component 216 may identify multiple documents or passages within documents that may be relevant as a potential answer. The predictions made by the extractive QA language model (implemented by language model component 216) may be evaluated via one or more evaluation models, such as exact match (EM) (i.e., measures the percentage of predictions that match any one of the ground truth answers exactly), F1 (i.e., the weighted average of Precision and Recall), span-F1, and/or span-EM, which generate scores for each prediction. The F1 and EM metrics measure the number of overlapping tokens between the predicted answers and the ground truth answers. F1 may be calculated as follows:

$$F 1 = 2 \times \frac{Precision \times Recall}{Precision + Recall}$$

Wherein precision is the ratio of the number of shared words to the total number of words in the prediction, and recall is the ratio of the number of shared words to the total number of words in the ground truth.

EM may be determined by evaluating whether characters of the model's prediction exactly match the characters of one of the true answers. In the event that the characters of the prediction match the characters of the true answers, then EM=1; and if there are no matches, EM=0.

Accordingly, the language model may assign a score to each document and/or passage, and the passage with the highest score may be returned as an answer to a user. Alternatively, if the language model does not find an answer, the language model may return notification indicative of such to a user.

Framework 200 may further include a fine-tuner 218. Server system 104 may fine-tune the language model (implemented by language model component 216) with fine-tuner 218 with target data, synthetic target data, and feedback provided by a user (e.g., a clinician operating user device(s) 102) by implementing one or more of layer freezing, and modifying a layer-wise learning rate. In some instances, fine tuning each of the language model with target data, may include training the entire language model based on a new/modified dataset. Here, the error is back-propagated through the entire architecture and the pre-trained weights of the model are updated based on the new dataset.

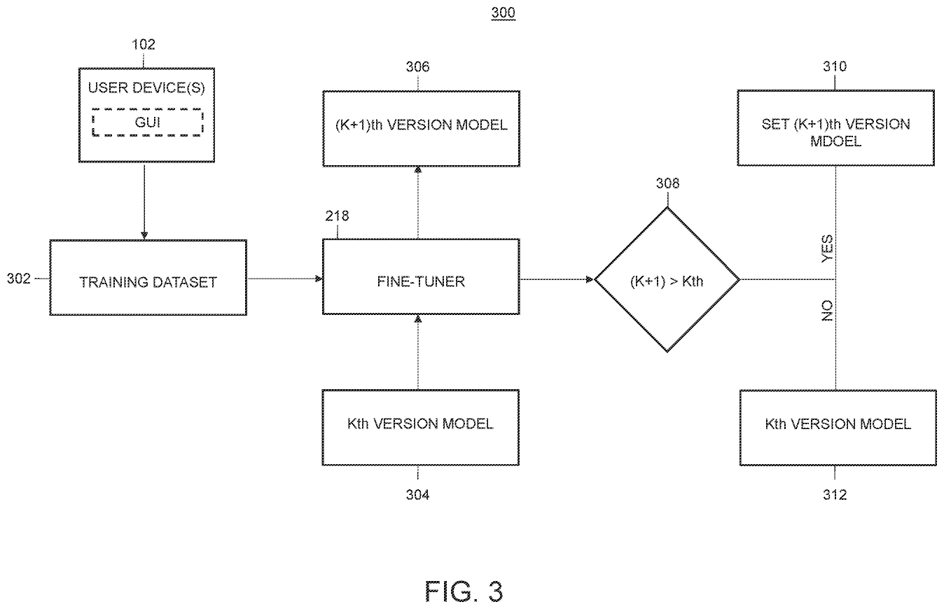

Referencing FIG. 3, a fine-tuner workflow 300 is depicted, according to one or more embodiments of the present disclosure. As depicted, the fine-tuner workflow 300 may include user device(s) 102, training dataset 302, and fine-tuner 218. User device(s) 102 may provide training dataset 302 with additional documents (e.g., clinical documents, images, and the like). The fine-tuner 218 may retrain a language model (e.g., an extractive QA model implemented by language model component 216) using feedback received from user device(s) or training dataset 302. As a result of the fine-tuning procedure, the weights of the original model are updated to account for the characteristics of training dataset 302 and the objects of the model.

For example, the fine-tuner 218 may receive a version of training dataset 302 that has been updated, and therefore update the parameter weights of the Kth version model 304 to create (K+1)th version model 306. If at 308 it is determined that the (K+1)th version model 306 version performs better than the Kth version model 304, then the (K+1)th version model 306 is set as the model that will be implemented 310. Alternatively, if the (K+1)th version model 306 does not perform better than the Kth version model, then the Kth version model continue to be used 312.

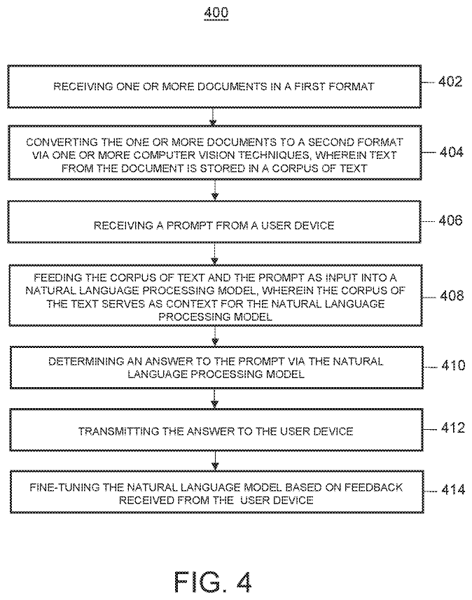

Referencing FIG. 4 a method for extractive question answering 400 is depicted, according to one or more embodiments of the present disclosure. At 402 server system 104 may receive a document in a first format. The document may be a clinical document (e.g., patient health records, patient charts, patient diagnosis, images associated with the patient) that initially includes protected health information. The document may be received over an encrypted network, for example network 108. Server system 104 may convert the document to a second format via one or more processes involving text extraction or OCR. Here, depending on the type of document that is received, one or more data and text extraction processes may be performed.

At 404 server system 104 may convert the document to a second format by redacting the protected health information included on the document. Here, depending on the type of document that is received one or more data and text extraction processes may be performed. For example, if the document is an image (e.g., an image of a lab result), the document may have its image data stored in a storage device (e.g., image data storage 206). The document may then be processed via OCR to identify text within the document. The identified text from the image may then be stored in a storage device (e.g., text data storage 210). Alternatively, if the document is a text-based document, the text may be extracted from the document and stored in a storage device (e.g., text data storage 210).

Step 404 may involve implementing one or more computer vision and natural language processing techniques to identify the protected health information, redact, remove, and/or obfuscate the protected health information. Here, the text is converted from its original form to a clean and sanitized version.

At 406 server system 104 may receive a prompt from a user device. For example, a user (e.g., a clinician) operating user device(s) 102 may submit a question (in real-time on-the-fly) or query to server system 104 relating to a medical task (e.g., a clinical analysis of a patient's lab results). As such, the clinician may submit a question using the clinician's natural language inquiring about what the patient's ICD code and diagnosis was on the lab result. Server system 104 may then attempt to predict an answer to the user's query based on the context made available to server system 104. In furtherance of attempting to generate an answer, server system 104 may tokenize and vectorize the text in the question.

At 408 server system 104 may feed the sanitized text and the prompt as input into a natural language processing model. For example, server system 104 may use the sanitized text as context and the user's question as input for an extractive language model that is capable of interpreting the user's natural language question; and identifying and providing an answer.

At 410 server system 104 may determine an answer to the prompt via the natural language processing model. Server system 104 may leverage one or more information retrieval modules that identify documents and passages within documents that may contain the answer to the user's question. For example, in the instance that the server system 104 receives an inquiry about what the patient's ICD code and diagnosis was on the lab result as discussed at 406, one or more retrieval models associated with extractive QA model may parse through the sanitized text to identify words, numbers, formulas, etc. that may answer the clinician's inquiry. Server system 104 may evaluate the tokens and vectors of the question text generated at 406, and further leverage one or more models such as BERT, RoBERTA, and/or GPT2 to identify passages that may contain the answer. Each identified passage and document that may have an answer, may be scored according to one or more metrics (e.g., via one or more EM, F1, span-EM, and/or span F1 metrics).

At 412 server system 104 may transmit the answer to the user device. Here, server system may transmit the answer (which includes the specific passage within the document that includes the answer) and a confidence score (that represents the language model's certainty that the provided answer is accurate). The transmission of the answer may include instructions to dynamically modify an interactive GUI of the user device. The transmission of the answer may additionally include instructions that cause the answer to be presented in text form, with the relevant span of text including the answer being highlighted. For example, server system 104 may transmit an answer and a confidence score to user device(s) 102, in response to a user (e.g., a clinician) submitting a question. The transmission by server system 104 may cause an interactive GUI on the user device(s) 102 to display a span of text including the answer to the clinician's question, wherein the answer itself is highlighted (in one or more colors) and/or emphasized (e.g., displayed in bold, italics, etc.). The transmission by server system 104 may additionally cause the interactive GUI to display a confidence score in the same or different region from where the answer is displayed. Following the example discussed at 410, here, server system 104 may transmit the lab result, with the span of text including the ICD code and patient diagnosis being highlighted, along with a confidence score that this answer is accurate. In addition, the transmission by server system 104 may cause the interactive GUI to display a feedback region, wherein a user operating user device(s) 102 can indicate whether the answer provided by the server system 104 is accurate and/or provides a relevant answer to the user's question.

At 414 server system 104 fine-tunes the natural language model based on feedback received from the user device. For example, in response to receiving an answer, a user operating user device(s) 102 may provide feedback regarding the provided answer via a region on the interactive GUI being displayed on user device(s) 102. In one instance, the feedback may be an acknowledgement that the answer is correct. In another instance, the feedback may be an indication that there may be a more accurate answer in another document. In another instance, the feedback provided by the clinician may indicate that the answer is incorrect. Notably, the feedback may be in the form of a text (e.g., a series of words or sentences, or data), numbers, formulas, and/or chemical compositions, entered by the user operating user device(s) 102. In another instance, the user may provide the feedback using speech and one or more speech recognition techniques are implemented to interpret the feedback. In one or more of the instances above, the feedback is transmitted to server system 104 and leveraged by the fine-tuner 218 to refine the language model implemented by language model component 216.

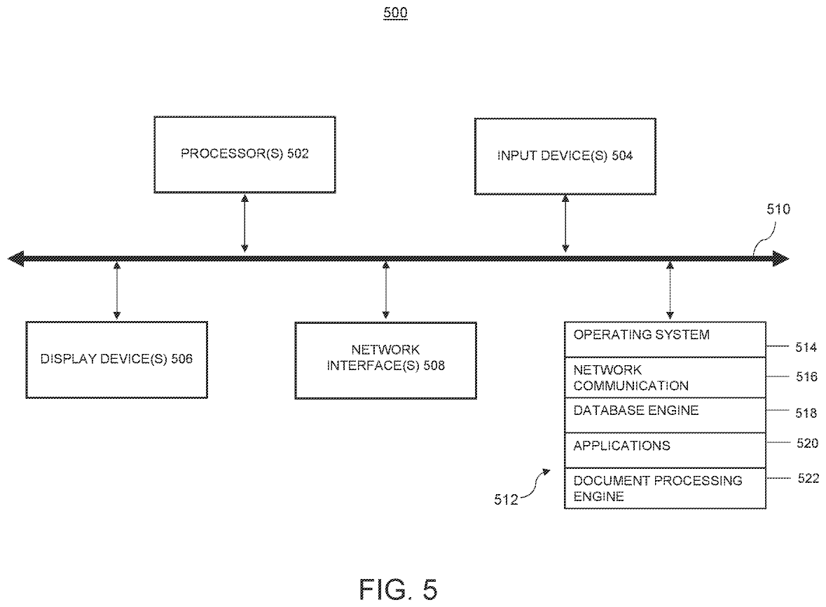

FIG. 5 illustrates a block diagram for a computing device, according to various embodiments of the present disclosure. For example, computing device 500 may function as server system 104. The computing device 500 may be implemented on any electronic device that runs software applications derived from compiled instructions, including without limitation personal computers, servers, smart phones, media players, electronic tablets, game consoles, email devices, etc. In some implementations, the computing device 500 may include processor(s) 502, (one or more) input device(s) 504, one or more display device(s) 506, one or more network interfaces 508, and one or more computer-readable medium(s) 512 storing software instructions. Each of these components may be coupled by bus 510, and in some embodiments, these components may be distributed among multiple physical locations and coupled by a network 108.

Display device(s) 506 may be any known display technology, including but not limited to display devices using Liquid Crystal Display (LCD) or Light Emitting Diode (LED) technology. Processor(s) 502 may use any known processor technology, including but not limited to graphics processors and multi-core processors. Input device(s) 504 may be any known input device technology, including but not limited to a keyboard (including a virtual keyboard), mouse, track ball, camera, and touch-sensitive pad or display. Bus 510 may be any known internal or external bus technology, including but not limited to industry standard architecture (ISA), extended industry standard architecture EISA, peripheral component interconnect (PCI), peripheral component interconnect (PCI) Express, universal serial bus (USB), Serial advanced technology attachment (ATA), or FireWire. Computer-readable medium(s) 512 may be any non-transitory medium that participates in providing instructions to processor(s) 502 for execution, including without limitation, non-volatile storage media (e.g., optical disks, magnetic disks, flash drives, etc.), or volatile media (e.g., Synchronous dynamic random-access memory (SDRAM), read-only memory (ROM), etc.).

Computer-readable medium(s) 512 may include various instructions for implementing an operating system 514 (e.g., Mac OS®, Windows®, Linux). The operating system may be multi-user, multiprocessing, multitasking, multithreading, real-time, and the like. The operating system may perform basic tasks, including but not limited to: recognizing input from input device(s) 504; sending output to display device(s) 506; keeping track of files and directories on computer-readable medium(s) 512; controlling peripheral devices (e.g., disk drives, printers, etc.) which can be controlled directly or through an input/output (I/O) controller; and managing traffic on bus 510. Network communications instructions 516 may establish and maintain network connections (e.g., software for implementing communication protocols, such as TCP/IP, HTTP, Ethernet, telephony, etc.).

Database processing engine 518 may include instructions that enable computing device 500 to implement one or more methods as described herein. Application(s) 520 may be an application that uses or implements the processes described herein and/or other processes. The processes may also be implemented in operating system 514. For example, application(s) 520 and/or operating system 514 may execute one or more operations to intelligently process documents (i.e., clinical documents) via one or more natural language processing and/or machine learning algorithms.

Document processing engine 522 may be used in conjunction with one or more methods as described above. Upload documents (e.g., clinical documents) received at computing device 500 may be fed into document processing engine 522 to analyze and classify the documents and provide information and suggestions about the document to a user in real-time.

The described features may be implemented in one or more computer programs that may be executable on a programmable system including at least one programmable processor coupled to receive data and instructions from, and to transmit data and instructions to a data storage system (e.g., database(s) 106), at least one input device, and at least one output device. A computer program is a set of instructions that can be used, directly or indirectly, in a computer to perform a certain activity or bring about a certain result. A computer program may be written in any form of programming language (e.g., Janusgraph, Gremlin, Sandbox, SQL, Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random-access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation or a computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

It is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

What is claimed is:

1. A system comprising:
a server comprising one or more processors; and
a non-transitory memory, in communication with the server, storing instructions that when executed by the one or more processors, cause the one or more processors to implement a method comprising:
receiving one or more documents;
converting the one or more documents to a second format via one or more computer vision techniques, wherein text from the one or more documents is stored in a corpus of text;
receiving a prompt from a user device;
feeding the corpus of text and the prompt as input into a natural language processing model, wherein the corpus of text serves as context for the natural language processing model;
determining an answer to the prompt via the natural language processing model;
transmitting the answer to the user device; and
receiving feedback associated with the answer from the user device.

2. The system of claim 1, wherein converting the one or more documents to the second format further comprises redacting protected health information from the one or more documents.

3. The system of claim 1, wherein the natural language processing model is an extractive QA model.

4. The system of claim 1, wherein the natural language processing model is pre-trained on electronic medical records.

5. The system of claim 1, wherein the natural language processing model is fine-tuned using feedback from the user device.

6. The system of claim 1, wherein determining the answer further comprises evaluating multiple possible answers included in the corpus of text via one or more metrics that:
compares the text in the prompt to the multiple possible answers to identify an exact match; or
applies a weighted average of precision and recall of each of the multiple possible answers.

7. The system of claim 1, wherein the transmitting the answer to the user device, further comprises generating instructions to visibly highlight and provide a passage of the one or more documents where the answer is located.

8. A computer-implemented method comprising:
receiving one or more documents;
converting the one or more documents to a second format via one or more computer vision techniques, wherein text from the one or more documents is stored in a corpus of text;
receiving a prompt from a user device;
feeding the corpus of text and the prompt as input into a natural language processing model, wherein the corpus of text serves as context for the natural language processing model;
determining an answer to the prompt via the natural language processing model;
transmitting the answer to the user device; and
receiving feedback associated with the answer from the user device.

9. The computer-implemented method of claim 8, wherein converting the one or more documents to the second format further comprises redacting protected health information from the one or more documents.

10. The computer-implemented method of claim 8, wherein the natural language processing model is an extractive question-answering model.

11. The computer-implemented method of claim 8, wherein the natural language processing model is pre-trained on electronic medical records.

12. The computer-implemented method of claim 8, wherein the natural language processing model is fine-tuned using feedback from the user device.

13. The computer-implemented method of claim 8, wherein determining the answer further comprises evaluating multiple possible answers included in the corpus of text via one or more metrics that:
compares the text in the prompt to the multiple possible answers to identify an exact match; or
applies a weighted average of precision and recall of each of the multiple possible answers.

14. The computer-implemented method of claim 8, wherein the transmitting of the answer to the user device further comprises generating instructions to visibly highlight and provide a passage of the one or more documents where the answer is located.

15. A non-transitory computer-readable medium storing instructions, that when executed by one or more processors, cause the one or more processors to implement the instructions for:
receiving one or more documents;
converting the one or more documents to a second format via one or more computer vision techniques, wherein text from the one or more documents is stored in a corpus of text;
receiving a prompt from a user device;
feeding the corpus of text and the prompt as input into a natural language processing model, wherein the corpus of text serves as context for the natural language processing model;
determining an answer to the prompt via the natural language processing model;
transmitting the answer to the user device; and
receiving feedback associated with the answer from the user device.

16. The non-transitory computer-readable medium of claim 15, wherein converting the one or more documents to the second format further comprises redacting protected health information from the one or more documents.

17. The non-transitory computer-readable medium of claim 15, wherein the natural language processing model is an extractive QA model.

18. The non-transitory computer-readable medium of claim 15, wherein the natural language processing model is pre-trained on electronic medical records.

19. The non-transitory computer-readable medium of claim 15, wherein the natural language processing model is fine-tuned using feedback from the user device.

20. The non-transitory computer-readable medium of claim 15, wherein determining the answer further comprises evaluating multiple possible answers included in the corpus of text via one or more metrics that:
compares the text in the prompt to the multiple possible answers to identify an exact match; or
applies a weighted average of precision and recall of each of the multiple possible answers.
